(19) United States
(12) Patent Application Publication
(10) Pub. No.: US 2005/0038042 A1
(43) Pub. Date: Feb. 17, 2005

(54) Modified release composition comprising a short-acting hypnotic for treatment of sleep disorders

(76) Inventors: Jenet Codd, Westmeath (IE); Alison Wilson, Westmeath (IE); Maurice Clancy, Kildare (IE); Melanie O'Hanlon, Offaly (IE)

Correspondence Address: Finnegan, Henderson, Farabow,;Garrett & Dunner, L.L.P., 1300 I Street, N.W., Washington, DC, 20005-3315, US

(21) Appl. No.: 10/704,633

(22) Filed: Nov. 12, 2003

Related U.S. Application Data
(60) Provisional application No. 60/426,369, filed on Nov. 15, 2002.

(51) Int. Cl. A61K 31/519

(52) U.S. Cl. 514/259.1

(57) ABSTRACT

A pharmaceutical composition comprising a first component comprising a short-acting hypnotic or a pharmaceutically acceptable salt thereof; a second component comprising at least one particle, wherein the at least one particle comprises a core and at least one coating over the core, the core comprising a short-acting hypnotic or a pharmaceutically acceptable salt thereof, and the at least one coating comprising at least one pharmaceutically acceptable polymer that is soluble at a pH greater than or equal to about 5.5, and optionally a third component comprising at least one particle, wherein the at least one particle comprises a core and at least one coating over the core, the core comprising a short-acting hypnotic or a pharmaceutically acceptable salt thereof, and the at least one coating comprising at least one pharmaceutically acceptable polymer that is soluble at a pH greater than or equal to about 6.0. Methods of treating sleep disorders, prolonging sleep, and inducing sleep using these compositions are also disclosed.

This application claims the benefit of U.S. Provisional Appliation No. 60/426,369, filed Nov. 15, 2002.

The present invention relates to a modified release composition comprising a short-acting hypnotic drug. In particular, the present invention relates to modified release compositions that in operation deliver a short-acting hypnotic in a pulsatile manner. The present invention further relates to solid oral dosage forms containing such a modified release composition. In addition, the present invention further relates to methods of treating sleep disorders in patients in need of such treatment by administering an effective amount of such compositions.

The plasma profile associated with the administration of a drug compound may be described as a “pulsatile profile” in which pulses of high active ingredient concentration, interspersed with low concentration troughs, are observed. A pulsatile profile containing two peaks may be described as “bimodal”. A pulsatile profile containing three peaks may be described as “trimodal”. Similarly, a composition or a dosage form that produces such a profile upon administration may be said to exhibit “pulsed release” of the active ingredient.

Conventional frequent dosage regimes in which an immediate release (IR) dosage form is administered at periodic intervals typically give rise to a pulsatile plasma profile. For instance, a peak in the plasma drug concentration is observed after administration of each IR dose with troughs (regions of low drug concentration) developing between consecutive administration time points. Such dosage regimes (and their resultant pulsatile plasma profiles) have particular pharmacological and therapeutic effects associated with them. For example, the wash out period provided by the fall off of the plasma concentration of the active ingredient between peaks has been thought to be a contributing factor in reducing or preventing patient tolerance to various types of drugs.

Many controlled release drug formulations are aimed at producing a zero-order release of the drug compound. Indeed, it is often a specific object of these formulations to minimize the peak-to-trough variation in drug plasma levels associated with conventional frequent dosage regimes. However, some of the therapeutic and pharmacological effects intrinsic in a pulsatile system may be lost or diminished as a result of the constant or nearly constant plasma levels achieved by zero-order release drug delivery systems. Thus, a modified release composition or formulation that substantially mimics the release of frequent IR dosage regimes, while reducing the need for frequent dosing, is desirable.

Controlled release is useful, for example, in the administration of short-acting hypnotic drugs for the treatment of sleep disorders. Short-acting hypnotics are compounds capable of inducing sedative, anxiolytic, myorelaxant, and anticonvulsive effects in mammals to which they are administered. Such compounds may also be useful in both inducing and prolonging sleep in mammals to which they are administered. Examples of such compounds include certain pyrazolopyrimidines, cyclopyrrolones, benzodiazepines, phenothiazines, and imidazopyridines.

Zaleplon, also known as N-[3-(3-cyanopyrazolo[1,5-a] pyrimidin-7-yl)phenyl]-N-ethylacetamide, is a novel pyrazolopyrimidine hypnotic that binds selectively to the benzodiazepine type I site on the GABA_{A} (γ-aminobutyric acid, type A) receptor complex.

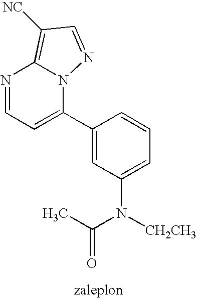

In animal models, it produced sedative, anxiolytic, myorelaxant, and anticonvulsive effects similar to those of marketed benzodiazepines. The results of clinical trials with zaleplon have shown it to be effective in shortening sleep onset with a more favorable safety profile than previously available benzodiazepine or nonbenzodiazepine hypnotics. Zaleplon was also found to have minimal effects on learning and memory and next-day residual (“hangover”) effects. Zaleplon is approved in the United States and Europe for insomnia in 5, 10 and 20 mg doses. It is marketed in the United States under the trademark Sonata® and is approved for the short-term treatment of insomnia. It has been shown to decrease the time to sleep onset for up to 28 days in controlled clinical studies. It may also be useful in providing sleep to those patients who have anxiety with insomnia.

In humans, zalepion has a time to maximum plasma concentration (tₘₐₓ) of 0.8 hours and a terminal half-life (t_{1/2}) in plasma of about 1 hour. These kinetic data predict a very fast onset and a short duration of action. Although zaleplon has been proven effective in treating patients suffering from certain sleep disorders, it has not been shown to consistently increase total sleep time or decrease the number of awakenings. This is primarily because the drug is rapidly metabolized to inactive metabolites, resulting in a mean duration of effect of 4-5 hours. Accordingly, an increased duration of effect, such as from 6-8 hours, while maintaining the advantages of no “hangover” effect, would be desirable to obtain.

Modified-release formulations comprising short-acting hypnotics are disclosed in EP 1064937A1, assigned to Sanofi-Synthelabo. This document relates to timed dual-release dosage forms of short-acting hypnotics. The first release, described as a “pulse” is an immediate release and the second “pulse” is a prolonged release over time. The invention relates to pellets, beads, granules, or spheroids coated with the drug and then optionally further coated with polymers, the solubility of which is pH-independent. The resultant release profiles of these formulations, however, provide the drug in an amount that continually increases over time until all of it is released. The disadvantage of such a release profile is that it is well known that short-acting hypnotics are subject to a first-pass metabolic effect in which the drug is rapidly metabolized to inactive metabolites. Using a prolonged release profile as described by the Sanofi-Synthelabo patent may potentially decrease the bioavailability of the drug as it is presented for metabolism at a relatively constant rate.

U.S. Pat. No. 6,228,398 B1 to Devane et al., assigned to the present assignee, also generally describes pharmaceutical formulations in which the active drug substance may be delivered in a pulsatile manner, such that there are two or more distinct phases of absorption of the drug after the administration of a single dose. Unlike the Sanofi-Synthelabo approach, this approach avoids the slow, constant release of drug substance that results in the metabolism problems discussed above. Devane, however, does not disclose the use of its compositions with zaleplon.

In U.S. Pat. Nos. 4,728,512, 4,794,001, and 4,904,476, (assigned on their face to American Home Products Corp.), are disclosed pharmaceutical formulations that provide three distinct releases of drug substance from a single dose. The formulations contain three groups of spheroids containing an active medicinal substance; the first group of spheroids is uncoated and rapidly disintegrates upon ingestion to release an initial dose of medicinal substance. The second group of spheroids is coated with a pH-sensitive coat to provide a second dose; and the third group of spheroids is coated with a pH-independent coat to provide a third dose.

The disadvantages associated with the prolonged release that may occur with the use of, among other things, pH-independent coatings, may be overcome if one were to administer short-acting hypnotics, such as zaleplon, using a formulation that presents the drug in a pulsatile manner such that the plasma concentration of the drug increases quickly (“pulses”) several times over a desired period of time. Such a release profile would have the advantages of decreasing the amount of drug that is metabolized, would allow for prolonged therapeutic effects with a single dose as opposed to formulations currently available, and would be suitable for chronic dosing. Furthermore, presentation of the drug in the form of small pellets or minitablets would have the advantage of ease of dosing for patients that cannot tolerate oral administration of tablets or capsules now available. Such pellets or minitablets could be easily taken with a meal, for example, by sprinkling them on food.

The inventors have accordingly developed methods to prepare and use multiparticulate modified release compositions that deliver short-acting hypnotics in a pulsatile manner to overcome one or more of the problems discussed above associated with currently-known compositions containing these compounds.

Specifically, the invention relates to a method for treating sleep disorders by administering to patients in need of such treatment an effective amount of a pharmaceutical composition comprising:

a) a first component comprising a short-acting hypnotic or a pharmaceutically acceptable salt thereof;

b) a second component comprising at least one particle, wherein the at least one particle comprises a core and at least one coating over the core, the core comprising a short-acting hypnotic or a pharmaceutically acceptable salt thereof, and the at least one coating comprising at least one pharmaceutically acceptable polymer that is soluble at a pH greater than or equal to about 5.5, such as 6.0 or, further, such as 7.0; and optionally

c) a third component comprising at least one particle, wherein the at least one particle comprises a core and at least one coating over the core, the core comprising a short-acting hypnotic or a pharmaceutically acceptable salt thereof, and the at least one coating comprising at least one pharmaceutically acceptable polymer that is soluble at a pH greater than or equal to about 5.5, such as 6.0 or, further, such as 7.0.

In one embodiment, the short-acting hypnotic in the first, second, and the optional third components is zaleplon.

As will be discussed further below, the pH-dependent nature of the at least one polymer comprising the coating(s) allows the release of the the short-acting hypnotic, such as zaleplon, to be controlled.

In another aspect, the present invention relates to a pharmaceutical composition comprising

a) a first component comprising a short-acting hypnotic or a pharmaceutically acceptable salt thereof; and

b) a second component comprising at least one particle, wherein the at least one particle comprises a core and at least one coating over the core, the core comprising a short-acting hypnotic or a pharmaceutically acceptable salt thereof, and the at least one coating comprising at least one pharmaceutically acceptable polymer that is soluble at a pH greater than or equal to about 5.5, such as 6.0, or further such as 7.0.

In one embodiment, the short-acting hypnotic in the first and second components is zaleplon.

In yet another aspect, the present invention relates to a pharmaceutical composition comprising

a) a first component comprising a short-acting hypnotic or a pharmaceutically acceptable salt thereof;

b) a second component comprising at least one particle, wherein the at least one particle comprises a core and at least one coating over the core, the core comprising a short-acting hypnotic or a pharmaceutically acceptable salt thereof, and the at least one coating comprising at least one pharmaceutically acceptable polymer that is soluble at a pH greater than or equal to about 5.5; and

c) a third component comprising at least one particle, wherein said at least one particle comprises a core and at least one coating over said core, said core comprising a short-acting hypnotic or a pharmaceutically acceptable salt thereof, and said at least one coating comprising at least one pharmaceutically acceptable polymer that is soluble at a pH greater than or equal to about 6.0. Alternatively, the third component may comprise a coating comprising at least one pharmaceutically acceptable polymer that is soluble at a pH greater than or equal to about 7.0.

In one embodiment, the short-acting hypnotic in each of the three components is zaleplon.

In yet another aspect, the present invention relates to a pharmaceutical composition comprising

a) a first component comprising a short-acting hypnotic or a pharmaceutically acceptable salt thereof;

b) a second component comprising at least one particle, wherein the at least one particle comprises a core and at least one coating over the core, the core comprising a short-acting hypnotic or a pharmaceutically acceptable salt thereof, and the at least one coating comprising at least one pharmaceutically acceptable polymer that is soluble at a pH greater than or equal to about 6.0; and

c) a third component comprising at least one particle, wherein said at least one particle comprises a core and at least one coating over said core, said core comprising a short-acting hypnotic or a pharmaceutically acceptable salt thereof, and said at least one coating comprising at least one pharmaceutically acceptable polymer that is soluble at a pH greater than or equal to about 7.0.

In one embodiment, the short-acting hypnotic in each of the three components is zaleplon.

In another embodiment, the present invention provides method of treating sleep disorders, inducing sleep, and prolonging the sleep of patients in need of such treatment by administration of an effective amount of such a formulation to the patient.

### BRIEF DESCRIPTION OF THE DRAWINGS

FIG. 1 is the dissolution profile of beads as prepared in Examples 1 and 2 in 0.01N hydrochloric acid solution.

FIG. 2 is the dissolution profile of beads as prepared in Examples 3, 4, and 5, measured for 2 h in 0.01N hydrochloric acid, followed by pH 7.2 phosphate buffer for additional 4 h.

FIG. 3 is the dissolution profile of capsules as prepared in Examples 6 and 7, measured in 0.01N hydrochloric acid for 2 h, followed by pH 7.2 phosphate buffer for an additional 4 h.

FIG. 4 is the dissolution profile of tablets as prepared in Examples 10 and 11, measured in 0.01N hydrochloric acid.

FIG. 5 is the dissolution profile for tablets as prepared in Examples 12, 13, and 14, measured in 0.01N hydrochloric acid for 2 h, followed by pH 7.2 phosphate buffer for an additional 4 h.

FIG. 6 is the dissolution profile of capsules as prepared in Examples 15 and 16, measured in 0.01N hydrochloric acid for 2 h, followed by pH 7.2 phosphate buffer for an additional 4 h.

The term “short-acting hypnotic,” as used herein, refers to compounds capable of inducing sedative, anxiolytic, myorelaxant, and anticonvulsive effects in mammals to which they are administered. Among the short-acting hypnotics that may be used according to the present invention include, but are not limited to, pyrazolopyrimidines (such as zaleplon), cyclopyrrolones (such as zopiclone and its enantiomers, such as (R)-zopliclone), benzodiazepines (such as triazolam, temazepam, and brotizolam), phenothiazines (such as alimemazine or the tartrate salt thereof), and imidazopyridines (such as zolpidem).

The term “zaleplon” as used herein in relation to the compositions according to the invention means N-[3-(3-cyanopyrazolo[1,5-a] pyrimidin-7-yl)phenyl]-N-ethylacetamide, or its pharmaceutically acceptable salts.

The term “zopiclone” as used herein in relation to the compositions according to the invention means 6-(5-chloro-2-pyridinyl)-6,7-dihydro-7-oxo-5H-pyrrolo[3,4-b]pyrazin-5-yl-1-piperazinecarboxylate.

The term “triazolam” as used herein in relation to the compositions according to the invention means 8-chloro-6-(o-chlorophenyl )-1-methyl-4H-s-triazolo-(4,3-alpha)(1,4) benzodiazepine.

The term “temazepam” as used herein in relation to the compositions according to the invention means 7-chloro-1,3-dihydro-3-hydroxy-1-methyl-5-phenyl-2H-1,4-benzodiaz epin-2-one.

The term “brotizolam” as used herein in relation to the compositions according to the invention means 2-bromo-4-(o-chlorophenyl)-9-methyl-6H-thieno[3,2-f]-s-triazolo[4,3-a][1,4]diazepine.

The term “alimemazine” as used herein in relation to the compositions according to the invention means N,N-dimethyl-2-[(phenothiazin-10-yl )methyl] propylamine hemitartrate.

The term “zolpidem” as used herein in relation to the compositions according to the invention means N, N,6-trimethyl-2-p-toyl-imidazo(1,2,-a)pyridine-3-acetamide L-(+)-tartrate (2:1).

The term “multiparticulate” as used herein means a plurality of discrete or aggregated particles, beads, pellets, granules, tablets, or mixture thereof without regard to their size, shape, or morphology.

The term “sleep disorders” as used herein means disorders related to falling asleep and staying asleep, such as, for example, chronic insomnia, irregular sleep-wake schedules, rotating shift work where a regular sleep schedule cannot be maintained, jet-lag, in depression and other psychological ailments, and other medical conditions where disturbed sleep is an issue. The term “insomnia” is used to describe all conditions related to the perception by the patient of inadequate or non-restful sleep. Sleep disorders are among the most common symptoms found in general medical practice. Insomnia is a frequent complaint, being reported by 13% to 45% of the adult population. Symptoms include frequent or continuous difficulty in falling asleep at night, frequent nocturnal awakenings, and/or early morning awakenings. Sleeplessness itself may take many forms, but it appears to be most closely related to age, sex, and the individual's psychopathological status, and is of particular importance in older persons and in women. Therefore, the treatment of sleep disorders can include both inducing and prolonging the sleep of patients in need thereof.

The term “modified release” as used herein means release which is not immediate release and encompasses controlled release, sustained release and delayed release.

As used herein, the term “pharmaceutically acceptable salt” includes salts that are physiologically tolerated by a subject. Such salts are typically prepared from a suitable inorganic and/or organic acid and a suitable basic compound. Examples of suitable inorganic acids include, but are not limited to, hydrochloric, hydrobromic, hydroiodic, nitric, sulfuric, and phosphoric acid. Organic acids may be aliphatic, aromatic, carboxylic, and/or sulfonic acids. Suitable organic acids include, but are not limited to, formic, acetic, propionic, succinic, camphorsulfonic, citric, fumaric, gluconic, lactic, malic, mucic, tartaric, para-toluenesulfonic, glycolic, glucuronic, maleic, furoic, glutamic, benzoic, anthranilic, salicylic, phenylacetic, mandelic, pamoic, methanesulfonic, ethanesulfonic, pantothenic, benzenesulfonic (besylate), stearic, sulfanilic, alginic, galacturonic, p-bromophenylsulfonic, camphorsulfonic, carbonic, ethanesulfonic, gluconic, isethionic, maleicmandelic, oxalic, pantothenic, p-toluenesulfonic and the like. Examples of such pharmaceutically acceptable salts of zaleplon, thus, include, but are not limited to, acetate, benzoate, .beta.-hydroxybutyrate, bisulfate, bisulfite, bromide, butyne-1,4-dioate, caproate, chloride, chlorobenzoate, citrate, dihydrogenphosphate, dinitrobenzoate, fumarate, glycollate, heptanoate, hexyne-1,6-dioate, hydroxybenzoate, iodide, lactate, maleate, malonate, mandelate, metaphosphate, methanesulfonate, methoxybenzoate, methylbenzoate, monohydrogenphosphate, naphthalene-1-sulfonate, naphthalene-2-sulfonate, oxalate, phenylbutyrate, phenylproionate, phosphate, phthalate, phylacetate, propanesulfonate, propiolate, propionate, pyrophosphate, pyrosulfate, sebacate, suberate, succinate, sulfate, sulfite, sulfonate, tartrate, xylenesulfonate, and the like.

As used herein, the term “pharmaceutically acceptable excipient” includes compounds that are compatible with the other ingredients in a pharmaceutical formulation and not injurious to the subject when administered in therapeutically effective amounts.

The proportion of the short-acting hypnotic, such as zaleplon, as active ingredient contained in each component may be the same or different depending on the desired dosing regime. For example, in the bipulsatile formulations, each component may contain the active ingredient, such as zaleplon, in an amount in the range of about 30% to about 70%. In the tripulsatile formulations, each component may contain the active ingredient, such as zaleplon, in an amount in the range of about 15% to about 50%. The active ingredient may be present, in the first component individually or in combination with the active ingredient (or active ingredients) in the second component, in any amount sufficient to elicit a therapeutic response.

The short acting hypnotics contained in any of the above-described compositions may be in present in an amount such that the total dose administered is an of from about 0.1 mg to about 100 mg. The selection of a suitable dose of the short-acting hypnotics according to the present invention will depend on factors associated with each individual patient and the most appropriate dose may be selected using knowledge known to those skilled in the art

Zaleplon may be present in any of the above-described compositions such that the total dose administered is an amount of from about 5 mg to about 60 mg, such as from about 1 mg to 30 mg, from about 5 mg to about 30 mg; for example, about 5 mg, about 10 mg, about 15 mg, or about 20 mg. The dose selected will depend on factors associated with each individual patient and the most appropriate dose may be selected using knowledge known to those skilled in the art. In any event, it is desired that the dose be selected such that the plasma concentration of zaleplon is in the range of about 10 ng/mL to 35 ng/mL over the time period in which is desired that zaleplon have a positive clinical effect. In addition, when it is desired to use a combination of pellets or tablets coated with pharmaceutically acceptable polymers, the solubility of which is pH dependent, the amount of zaleplon contained in each bead, and the number of such beads, may be determined by knowledge available to one skilled in the art.

Zaleplon may be prepared using methods described in U.S. Pat. No. 4,626,538, to Dusza et al. Zaleplon may also be prepared by methods well known to those skilled in the art.

In one embodiment, the formulations of the present invention are provided as membrane controlled formulations. The pH-dependent nature of the polymer(s) comprising the membrane, or coating, allows the release of the formulations to be controlled. Membrane controlled formulations of the present invention can be made by preparing a rapid release core, which may be a monolithic (e.g., tablet) or multi-unit (e.g., pellet) type, and coating the core with a coating comprising at least one polymer as discussed above.

In one embodiment, the short-acting hypnotic may be provided in a multiparticulate membrane controlled formulation. More specifically, the short-acting hypnotic may be formed into an active core by applying the drug to a nonpareil seed having an average diameter in the range of from about 0.4 to about 1.1 mm, such as from about 0.71 mm to about 0.85 mm. The short-acting hypnotic may be applied with or without additional excipients onto the inert cores, and may be sprayed from solution or suspension using a fluidized bed coater (e.g., Wurster coating) or pan coating system. Alternatively, the short-acting hypnotic may be applied as a powder onto the inert cores using a binder to bind the short-acting hypnotic onto the cores. Active cores may also be formed by extrusion of the core with suitable plasticizers (described below) and any other processing aids as necessary. Some of the short-acting hypnotic-containing cores are coated with at least one pharmaceutically acceptable polymer to form a membrane as discussed above, and others are left uncoated. The uncoated short-acting hypnotic-containing cores are thus an example of the first component of the inventive composition described above, i.e., an immediate release dosage form. The coated short-acting hypnotic containing cores are thus an example of the second and third components of the inventive composition, i.e., the pH-dependent coating allows modified release so that when the immediate release and modified release components are combined in the inventive composition, the envisioned bipulsatile or tripulsatile release of the short-acting hypnotic may be achieved.

In another embodiment, the short-acting hypnotic may be provided in a multiparticulate membrane controlled formulation, so-called minitablets, in which it is formed into an active core comprising the short-acting hypnotic and optionally other ingredients, suitable excipients for example, by direct compression or granulation. This active core may then be coated with an appropriate membrane coating. The immediate release dosage minitablets are not coated with a membrane coating as described. Such minitablets may have a diameter in the range of about 1.5 mm to about 6 mm.

The at least one pharmaceutically acceptable polymer applied as a membrane coating to the drug-containing cores may be chosen from, for example, polyvinyl alcohol, polyvinylpyrrolidone, methylcellulose, hydroxypropylcellulose, hydroxypropylmethyl cellulose, hydroxypropyl methylcellulose phathallate (including HPMCP 50 and HPMCP 55), polyethylene glycol, EUDRAGIT™ polymers and/or mixtures thereof. EUDRAGIT™ polymers (available from Rohm Pharma) are polymeric lacquer substances based on acrylates and/or methacrylates. Suitable EUDRAGIT™ polymers which are slightly permeable to the active ingredient and water, and exhibit a pH-dependent permeability include, but are not limited to, EUDRAGIT™ L and EUDRAGIT™ S.

Methacrylic acid co-polymers such as EUDRAGIT™ S and EUDRAGIT™ L (Rohm Pharma) are particularly suitable for use in the controlled release formulations of the present invention. These polymers are gastro-resistant and enterosoluble polymers. Their polymer films are insoluble in pure water and diluted acids. When such polymer films become soluble is a function of the pH of the environment as well as the content of carboxylic acid monomer used to make the polymer. EUDRAGIT™ S and EUDRAGIT™ L can be used individually in the polymer coating or in combination in any ratio. By using a combination of the polymers, the polymeric material may exhibit a solubility at a pH between the pHs at which EUDRAGIT™ L and EUDRAGIT™ S are separately soluble.

EUDRAGIT™ L is an anionic polymer synthesized from methacrylic acid and methacrylic acid methyl ester. It is insoluble in acids and pure water. It becomes soluble in neutral to weakly alkaline conditions. The permeability of EUDRAGIT™ L is pH dependent. Among the EUDRAGIT™ L polymers which are suitable for use in the present invention are EUDRAGIT™ L 100-55, EUDRAGIT™ L 100, EUDRAGIT™ L 30 D-55 and EUDRAGIT™ L 12.5. Membranes comprising EUDRAGIT™ L 100-55 and EUDRAGIT™ L 30 D-55 become increasingly permeable at a pH greater than or equal to about 5.5. Membranes comprising EUDRAGIT™ L 100 and EUDRAGIT™ L 12.5 become increasingly permeable at a pH greater than or equal to about 6.0.

EUDRAGIT™ S is an anionic polymer synthesized from methacrylic acid and methacrylic acid methyl ester. It is insoluble in acids and pure water. It becomes soluble in neutral to weakly alkaline conditions. The permeability of EUDRAGIT™ S is pH dependent. Above pH 7.0, the polymer becomes increasingly permeable. Among the EUDRAGIT™ S polymers which are suitable for use in the present invention are EUDRAGIT™ S 100 and EUDRAGIT™ S 12.5.

The coating may also include one or more auxiliary agents such as fillers, plasticizers, and/or anti-foaming agents. Representative fillers include talc, fumed silica, glyceryl monostearate, magnesium stearate, calcium stearate, kaolin, colloidal silica, gypsum, micronized silica, and magnesium trisilicate. The quantity of filler used typically ranges from about 2% to about 300% by weight, and can range from about 20 to about 100%, based on the total dry weight of the polymer. In one embodiment, talc is the filler.

The coating membranes, and functional coatings as well, can also include a material that improves the processing of the polymers. Such materials are generally referred to as plasticizers and include, for example, adipates, azelates, benzoates, citrates, isoebucates, phthalates, sebacates, stearates and glycols. Representative plasticizers include acetylated monoglycerides, butyl phthalyl butyl glycolate, dibutyl tartrate, diethyl phthalate, dimethyl phthalate, ethyl phthalyl ethyl glycolate, glycerin, ethylene glycol, propylene glycol, triacetin citrate, triacetin, tripropinoin, diacetin, dibutyl phthalate, acetyl monoglyceride, polyethylene glycols, castor oil, triethyl citrate, polyhydric alcohols, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate, glyceryl monocaprylate, and glyceryl monocaprate. In one embodiment, the plasticizer is dibutyl sebacate. The amount of plasticizer used in the polymeric material typically ranges from about 10% to about 50%, for example, about 10, 20, 30, 40, or 50%, based on the weight of the dry polymer.

Anti-foaming agents, for example simethicone, can also be included. The amount of anti-foaming agent used typically comprises from about 0% to about 0.5% of the final formulation.

The amount of polymer to be used in the membrane controlled formulations is typically adjusted to achieve the desired drug delivery properties, including the amount of drug to be delivered, the rate and location of drug delivery, the time delay of drug release, and the size of the multiparticulates in the formulation. The amount of polymer applied typically provides an about 2% to about 50% weight gain to the cores. In one embodiment, the weight gain from the polymeric material ranges from about 3% to about 30%.

The combination of all solid components of the coating material, including co-polymers, fillers, plasticizers, and optional excipients and processing aids, typically provides an about 3% to about 60% weight gain on the cores. In one embodiment, the weight gain is about 3% to about 45%.

The coating material can be applied by any known method, for example, by spraying using a fluidized bed coater (e.g., Wurster coating) or pan coating system. Coated cores are typically dried or cured after application of the polymeric material. Curing means that the multiparticulates are held at a controlled temperature for a time sufficient to provide stable release rates. Curing can be performed, for example, in an oven or in a fluid bed drier. Curing can be carried out at any temperature above room temperature.

A sealant or barrier can also be applied to the polymeric coating. A sealant or barrier layer may also be applied to the core prior to applying the polymeric material. A sealant or barrier layer is not intended to modify the release of the short-acting hypnotic. Suitable sealants or barriers are permeable or soluble agents such as hydroxypropyl methylcellulose, hydroxypropyl cellulose, hydroxypropyl ethylcellulose, and xanthan gum.

Other agents can be added to improve the processability of the sealant or barrier layer. Such agents include talc, colloidal silica, polyvinyl alcohol, titanium dioxide, micronized silica, fumed silica, glycerol monostearate, magnesium trisilicate and magnesium stearate, or a mixture thereof. The sealant or barrier layer can be applied from solution (e.g., aqueous) or suspension using any known means, such as a fluidized bed coater (e.g., Wurster coating) or pan coating system. Suitable sealants or barriers include, for example, OPADRY WHITE Y-1-7000 and OPADRY OY/B/28920 WHITE, each of which is available from Colorcon Limited, England.

In one embodiment is provided a so-called bipulsatile formulation, comprising a total dose of 20 mg of zaleplon, administered as a mixture of first and second pellets. The first pellets comprise a core of 10 mg of zaleplon, and may optionally further comprise appropriate additives or excipients, on non pareil seeds. The second pellets comprise a core of 10 mg of zaleplon on non pareil seeds, and may optionally further comprise appropriate additives or excipients, and further comprise a coating of EUDRAGIT L. Such a formulation may be administered orally in the form of a capsule containing said first and second pellets. The proportion of first and second pellets to be administered in such a formulation may be determined by those skilled in the art without undue experimentation. In such a formulation, the weight of the coating of EUDRAGIT L, with respect to the uncoated drug loaded core, may be in the range from about 1% to about 50%, or in the range from about 1% to about 30%, or in the range from about 1% to about 20% or may be in the range from about 2% to about 10%, or may be about 5%, by weight. The desired weight of each coating will depend on the desired release and pharmacokinetic profile and may be determined by one skilled in the art without undue experimentation.

In another embodiment is provided a bipulsatile formulation comprising a total dose of 20 mg of zaleplon, administered as a mixture of first and second minitablets. The first minitablets comprise a core of 10 mg of zaleplon, and may optionally further comprise any appropriate additives or excipients, said minitablets produced by direct compression or granulation of the zaleplon and other optional ingredients. The second minitablets comprise a core of 10 mg of zaleplon, and may optionally further comprise any appropriate additives or excipients, and further comprising a coating of EUDRAGIT L. Such a formulation may be administered orally in the form of a capsule containing said first and second minitablets. The proportion of first and second minitablets to be administered in such a formulation may be determined by those skilled in the art without undue experimentation. In such a formulation, the weight of the coating of EUDRAGIT L with respect to the uncoated drug loaded core may be in the range from about 1% to about 50%, or in the range from about 1% to about 30%, or in the range from about 1% to about 20% or may be in the range from about 2% to about 20%. The desired weight of each coating will depend on the desired release and pharmacokinetic profile and may be determined by one skilled in the art without undue experimentation.

In another embodiment is provided a so-called tripulsatile formulation comprising a total dose of 20 mg of zaleplon, administered as a mixture of first, second, and third pellets. The first pellets comprise a core of 6.7 mg of zaleplon, and may optionally further comprise appropriate additives or excipients, on non pareil seeds. The second pellets comprise a core of 6.7 mg of zaleplon on non pareil seeds, and may optionally further comprise appropriate additives or excipients, and further comprise a coating of EUDRAGIT L. The third pellets comprise a core of 6.7 mg of zaleplon on non pareil seeds, and may optionally further comprise appropriate additives or excipients, and further comprise a coating of EUDRAGIT S. Such a formulation may be administered orally in the form of a capsule containing said first, second, and third pellets. The proportion of first, second, and third pellets to be administered in such a formulation may be determined by those skilled in the art without undue experimentation. In such a formulation, the weight of the coating of EUDRAGIT L and Eudragit S with respect to the uncoated drug loaded core independently may be in the range from about 1% to about 50%, or in the range from about 1% to about 30%, or in the range from about 1% to about 20%, or may be in the range from about 2% to about 10%, or may be about 4 to about 5%, by weight. The desired weight of each coating will depend on the desired release and pharmacokinetic profile and may be determined by one skilled in the art without undue experimentation.

In another embodiment is provided a tripulsatile formulation comprising a total dose of 20 mg of zaleplon, administered as a mixture of first, second, and third minitablets. The first minitablets comprise a core of 6.7 mg of zaleplon, and may optionally further comprise any appropriate additives or excipients, said minitablets produced by direct compression or granulation of the zaleplon and other optional ingredients. The second minitablets comprise a core of 6.7 mg of zaleplon, and may optionally further comprise any appropriate additives or excipients, and further comprising a coating of EUDRAGIT L. The third minitablets comprise a core of 6.7 mg of zaleplon, and may optionally further comprise any appropriate additives or excipients, and further comprising a coating of EUDRAGIT S. Such a formulation may be administered orally in the form of a capsule containing said first, second, and third minitablets. The proportion of first, second, and third minitablets to be administered in such a formulation may be determined by those skilled in the art without undue experimentation. In such a formulation, the weight of the coating of EUDRAGIT L and Eudragit S with respect to the uncoated drug loaded core may independently be in the range from about 1% to about 50%, or in the range from about 1% to about 30%, or in the range from about 2% to about 20%. The desired weight of each coating will depend on the desired release and pharmacokinetic profile and may be determined by one skilled in the art without undue experimentation.

Any of the pharmaceutical compositions and dosage forms described herein may further comprise one or more pharmaceutically active compounds other than zaleplon. Such compounds may be included to treat, prevent, and/or manage the same condition being treated, prevented, and/or managed with zaleplon, or a different one. Compounds that may be suitable for such purpose include, but are not limited to, zopiclone, triazolam, temazepam, brotizolam, alimemazine, and zolpidem. Those of skill in the art are familiar with examples of the techniques for incorporating additional active ingredients into compositions comprising zaleplon. Alternatively, such additional pharmaceutical compounds may be provided in a separate formulation and co-administered to a subject with a zaleplon composition according to the present invention. Such separate formulations may be administered before, after, or simultaneously with the administration of the zaleplon compositions of the present invention.

### EXAMPLES

The examples that follow are intended to be illustrative but not limiting of the

### Example 1

Immediate Release Beads Containing Zaleplon

1350 g of non pareil seeds (0.71-0.85 mm) were coated with a suspension of the following composition.

| | | |
|---|---|---|
| Zaleplon (micronised) | 20.0% | 122.34 g |
| Hydroxypropyl Methylcellulose 3 cps | 7.4% | 45.27 g |
| Polysorbate 80 | 0.2% | 1.22 g |
| Colloidal Silicon Dioxide | 1.7% | 10.40 g |
| Water USP | 70.7% | 432.47 g |

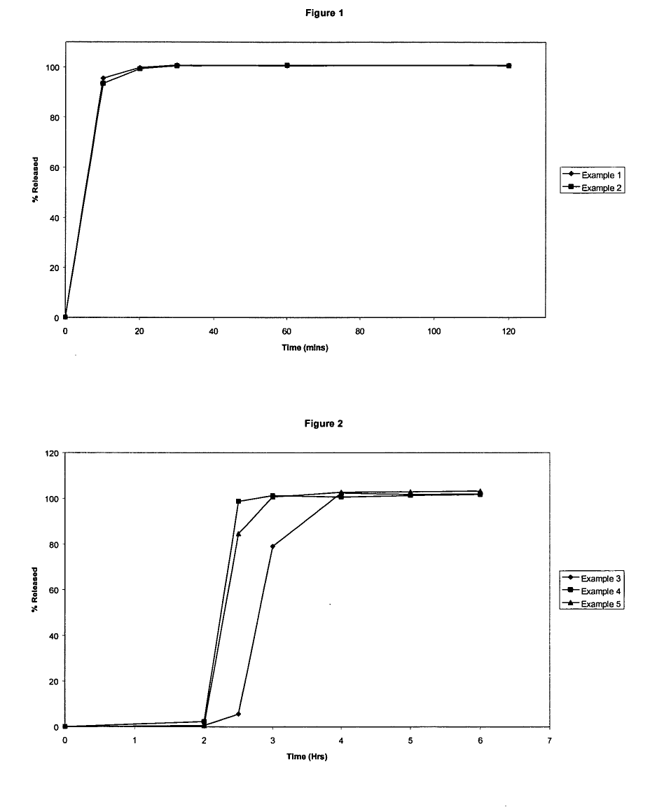

The coating was performed by Wurster application in a Uniglatt fluid bed processor (Glatt Protech, Leicester, England). The dissolution of the beads was measured using USP I (40 mesh) at a stirring speed of 100 rpm. The dissolution medium was 500 mL, 0.01M HCl at 37±0.5° C. The amount of dissolved zaleplon was measured by UV spectrophotometry at 232 nm. The dissolution curve is shown in FIG. 1.

### Example 2

Immediate Release Beads Containing Zaleplon

1250 g of non pareil seeds (0.71-0.85 mm) were coated with a suspension of the following composition.

| | | |
|---|---|---|
| Zaleplon (micronised) | 20.0% | 182.00 g |
| Hydroxypropyl Methylcellulose | 7.4% | 67.34 g |
| 3 cps | | |
| Polysorbate 80 | 0.2% | 1.82 g |
| Colloidal Silicon Dioxide | 1.7% | 15.47 g |
| Water USP | 70.7% | 643.37 g |

Coating was performed by Wurster application in a Uniglatt fluid bed processor (Glatt Protech, Leicester, England). The dissolution of the beads was measured using USP I (40 mesh) at a stirring speed of 100 rpm. Dissolution medium was 900 mL, 0.01M HCl at 37±0.5° C. The amount of dissolved zaleplon was measured by UV spectrophotometry at 232 nm. The dissolution curve is shown in FIG. 1.

### Example 3

Coated Beads

1000 g of Zaleplon immediate release beads from Example 1 were coated using a polymer suspension of the following composition.

| | | |
|---|---|---|
| Methacrylic Acid Copolymer | 50.00% | 320.0 g |
| Type B* | | |
| Dibutyl Sebecate | 1.25% | 8.0 g |
| Sterilised Talc | 2.50% | 16.0 g |
| Isopropyl Alcohol | 43.25% | 276.8 g |
| Water USP | 3.00% | 19.2 g |

*Eudragit S 12.5

Coating was performed using the Wurster process in a Glatt GPCG3 (Glaft Protech, Leicester, England). The dissolution of the beads was measured using USP I (40 mesh) at a stirring speed of 100 rpm. The dissolution test was performed in 500 mL, 0.01M HCl at 37±0.5° C. for 2 h. The beads were then transferred to 500 mL of Phosphate Buffer pH 7.2, and the dissolution test continued for a further 4 h. The amount of dissolved zaleplon was measured by UV spectrophotometry at 232 nm. The dissolution curve is shown in FIG. 2.

### Example 4

Coated Beads

1000 g of Zaleplon immediate release beads from Example 1 were coated using a polymer suspension of the following composition.

| | | |
|---|---|---|
| Methacrylic Acid Copolymer | 53.0% | 166.4 g |
| Type C* | | |
| Triethyl Citrate | 1.6% | 5.0 g |
| Sterilised Talc | 3.2% | 10.0 g |
| Water USP | 42.2% | 132.5 g |

*Eudragit L 30D 55

Coating was performed using the Wurster process in a Uniglatt (Glatt Protech, Leicester, England). The dissolution of the beads was measured as described in Example 3. The dissolution curve is shown in FIG. 2.

### Example 5

Coated Beads

1000 g of Zaleplon immediate release beads from Example 2 were coated using a polymer suspension of the following composition.

| | | |
|---|---|---|
| Methacrylic Acid Copolymer | 53.0% | 166.4 g |
| Type C* | | |
| Triethyl Citrate | 1.6% | 5.0 g |
| Sterilized Talc | 3.2% | 10.0 g |
| Water USP | 42.2% | 132.5 g |

*Eudragit L 30D 55

Coating was performed using the Wurster process in a Uniglatt fluid bed processor (Glatt Protech, Leicester, England). The dissolution of the beads was measured using USP I (40 mesh) at a stirring speed of 100 rpm. The dissolution test was performed in 900 mL, 0.01M HCl at 37±0.5° C. for 2 h. The beads were then transferred to 900 mL of Phosphate Buffer pH 7.2, and the dissolution test continued for a further 4 h. The amount of dissolved zaleplon was measured by UV spectrophotometry at 232 nm. The dissolution curve is shown in FIG. 2.

### Example 6

Extended Release Capsules

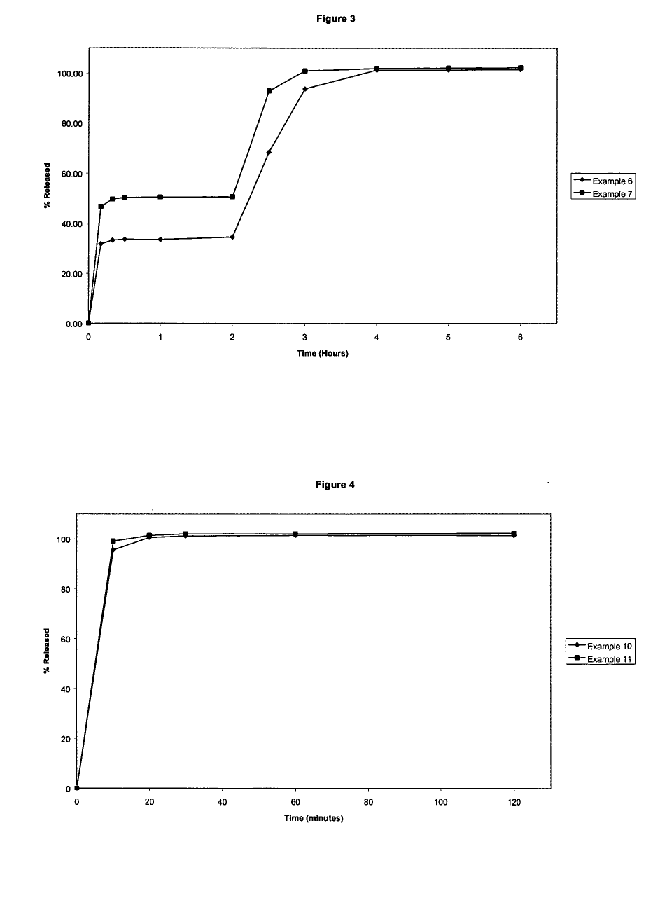

Capsules containing 85 mg of immediate release beads from Example 1, 95 mg of coated beads from Example 4 and 91 mg of coated beads from Example 3 are prepared using a Bosch encapsulator (Robert Bosch GmbH, Waiblingen, Germany). The total dose of Zaleplon is 20 mg. The dissolution of the capsules was simulated under conditions as found in Example 5. The simulated dissolution curve is shown in FIG. 3.

### Example 7

Extended Release Capsules

Capsules containing 83 mg of immediate release beads from Example 2 and 91 mg of coated beads from Example 5 were manufactured using a Bosch encapsulator (Robert Bosch GmbH, Waiblingen, Germany). The total dose of Zaleplon was 20 mg. The dissolution of the capsules was simulated under conditions as found in Example 5. The simulated dissolution curve is shown in FIG. 3.

### Example 8

Immediate Release Granule

An immediate release granule was prepared by top spray granulation in a Glatt GPCG3 (Glaft Protech, Leicester, England). An aqueous suspension containing Polyvinylpyrrolidone (Kollidon K30) and Sodium Lauryl Sulphate was applied to a mixture of Zaleplon, Sodium Starch Glycolate (Explotab) and Microcrystalline cellulose (Avicel PH101). The composition of the granule is as

| | | |
|---|---|---|
| Zaleplon (micronised) | 9.71% | 194.2 g |
| Microcrystalline Cellulose | 77.70% | 1554.0 g |
| Sodium Starch Glycolate | 3.88% | 77.6 g |
| Polyvinylpyrrolidone | 7.46% | 149.2 g |
| Sodium Lauryl Sulphate | 1.26% | 25.2 g |

### Example 9

Immediate Release Granule

An immediate release granule was prepared by top spray granulation in a Glatt GPCG3 (Glatt Protech, Leicester, England). An aqueous suspension containing Polyvinylpyrrolidone (Kollidon K30) and Sodium Lauryl Sulphate was applied to a mixture of Zaleplon, Sodium Starch Glycolate (Explotab) and Microcrystalline cellulose (Avicel PH101). The composition of the granule is as follows.

| | | |
|---|---|---|
| Zaleplon (micronised) | 15.14% | 302.8 g |
| Microcrystalline cellulose | 74.93% | 1498.5 g |
| Sodium Starch Glycolate | 4.04% | 80.8 g |
| Polyvinylpyrrolidone | 5.03% | 100.6 g |
| Sodium Lauryl Sulphate | 0.84% | 16.8 g |

### Example 10

Immediate Release Tablets

Immediate Release Tablets were prepared by blending Immediate Release Granule from Example 8 with Sodium Starch Glycolate, Colloidal Silicon Dioxide and Magnesium Stearate in a V cone Pharmatech Blender (Pharmatech Ltd., Warwicks, England). The tablets were compressed using a 10 station Piccola Tablet Press (Riva S.A., Buenos Aires, Argentina). The composition of the tablets is as follows.

| | | |
|---|---|---|
| Zaleplon (micronised) | 9.13% | 6.67 mg |
| Microcrystalline Cellulose | 73.04% | 53.36 mg |
| Sodium Starch Glycolate | 7.65% | 5.59 mg |
| Polyvinylpyrrolidone | 7.01% | 5.12 mg |
| Sodium Lauryl Sulphate | 1.18% | 0.86 mg |
| Colloidal Silicon Dioxide | 1.00% | 0.73 mg |
| Magnesium Stearate | 1.00% | 0.73 mg |

The dissolution of the tablets was measured as in Example 1. The dissolution curve is shown in FIG. 4.

### Example 11

Immediate Release Tablets

Immediate Release Tablets were prepared by blending Immediate Release Granule from Example 9 with Sodium Starch Glycolate, Colloidal Silicon Dioxide and Magnesium Stearate in a V cone Pharmatech Blender (Pharmatech Ltd., Warwicks, England). The tablets were compressed using a 10 station Piccola Tablet Press (Riva S.A., Buenos Aires, Argentina). The composition of the tablets is as follows.

| | | |
|---|---|---|
| Zaleplon (micronised) | 14.24% | 10.00 mg |
| Microcrystalline Cellulose | 70.43% | 49.46 mg |
| Sodium Starch Glycolate | 7.80% | 5.48 mg |
| Polyvinylpyrrolidone | 4.73% | 3.32 mg |
| Sodium Lauryl Sulphate | 0.79% | 0.55 mg |
| Colloidal Silicon Dioxide | 1.00% | 0.70 mg |
| Magnesium Stearate | 1.00% | 0.70 mg |

The dissolution of the tablets was measured as in Example 2. The dissolution curve is shown in FIG. 4.

### Example 12

Coated Tablets

600 g of immediate release tablets from Example 10 were coated in a Vector LCDS-3 Coater using a polymer suspension of the following composition.

| | | |
|---|---|---|
| Methacrylic Acid Copolymer | 50.00% | 192.00 g |
| Type B* | | |
| Dibutyl Sebecate | 1.25% | 4.80 g |
| Sterilised Talc | 2.50% | 9.60 g |
| Isopropyl Alcohol | 43.25% | 166.08 g |
| Water USP | 3.00% | 11.52 g |

*Eudragit S 12.5

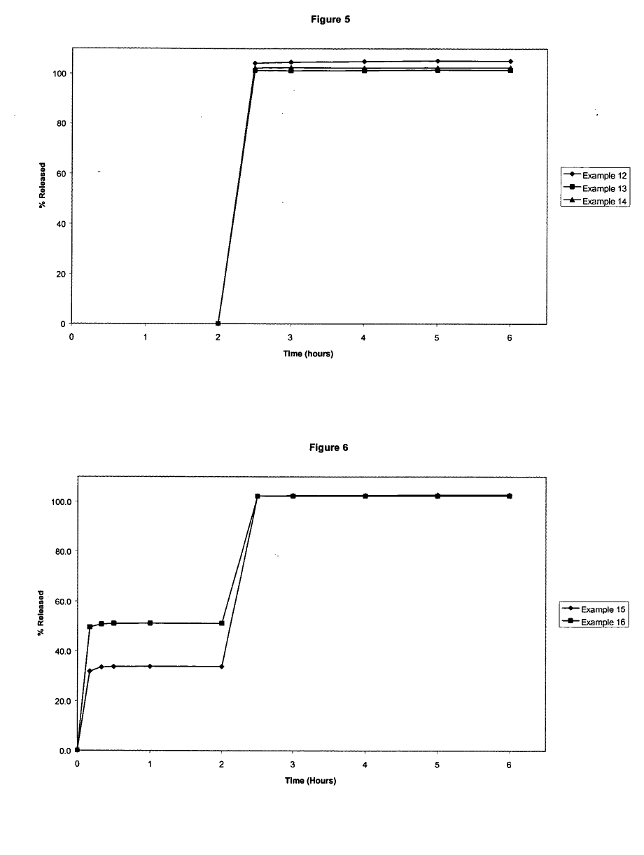

The dissolution of the tablets was measured as in Example 3. The dissolution curve is shown in FIG. 5.

### Example 13

Coated Tablets

600 g of immediate release tablets from Example 10 are coated in a Vector LCDS-3 Coater (Vector Corporation, Marion, Iowa, USA) using a polymer suspension of the following composition.

| | | |
|---|---|---|
| Methacrylic Acid Copolymer | 50.00% | 288.00 g |
| Type A* | | |
| Dibutyl Sebecate | 1.25% | 7.20 g |
| Sterilised Talc | 2.50% | 14.40 g |
| Isopropyl Alcohol | 43.25% | 249.12 g |
| Water USP | 3.00% | 17.28 g |

*Eudragit L 12.5

The dissolution of the tablets is simulated under conditions as found in Example 3. The simulated dissolution curve is shown in FIG. 5.

### Example 14

Coated Tablets

600 g of immediate release tablets from Example 11 are coated in a Vector LCDS-3 Coater (Vector Corporation, Marion, Iowa, USA) using a polymer suspension of the following composition.

| | | |
|---|---|---|
| Methacrylic Acid Copolymer | 50.00% | 288.00 g |
| Type A* | | |
| Dibutyl Sebecate | 1.25% | 7.20 g |
| Sterilised Talc | 2.50% | 14.40 g |
| Isopropyl Alcohol | 43.25% | 249.12 g |
| Water USP | 3.00% | 17.28 g |

*Eudragit L 12.5

The dissolution of the tablets is simulated under conditions as found in Example 5. The simulated dissolution curve is shown in FIG. 5.

### Example 15

Extended Release Capsules

Capsules are prepared manually containing one immediate release tablet from Example 10, one coated tablet from Example 12 and one coated tablet from Example 13. The total dose is 20 mg of zaleplon per capsule. The dissolution of the capsules is simulated under conditions as found in Example 5. The simulated dissolution curve is shown in FIG. 6.

### Example 16

Extended Release Capsules

Capsules are prepared manually containing one immediate release tablet from Example 11 and one coated tablet from Example 14. The total dose is 20 mg of Zaleplon per capsule. The dissolution of the capsules is simulated under conditions as found in Example 5. The simulated dissolution curve is shown in FIG. 6.

### Example 17

Administration of Extended Release Capsules to a Patient

An extended-release capsule, prepared as in Example 6, is administered to a non-elderly patient upon retiring at bedtime. The onset to sleep is observed to be in the range of about 20 to about 30 minutes. The duration of non-interrupted sleep is observed to be in the range of about 6 hours to about 8 hours.

### Example 18

Administration of Extended Release Capsules to a Patient

An extended-release capsule, prepared as in Example 15, is administered to a non-elderly patient upon retiring at bedtime. The onset to sleep is observed to be in the range of about 20 to about 30 minutes. The duration of non-interrupted sleep is observed to be in the range of about 6 hours to about 8 hours.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A pharmaceutical composition comprising:
a) a first component comprising zaleplon or a pharmaceutically acceptable salt thereof; and
b) a second component comprising at least one particle, wherein said at least one particle comprises a core and at least one coating over said core, said core comprising zaleplon or a pharmaceutically acceptable salt thereof, and said at least one coating comprising at least one pharmaceutically acceptable polymer that is soluble at a pH greater than or equal to about 5.5.

2. A pharmaceutical composition according to claim 1, wherein said at least one polymer is soluble at a pH greater than or equal to about 6.0.

3. A pharmaceutical composition according to claim 1, wherein said at least one polymer is soluble at a pH greater than or equal to about 7.0.

4. A pharmaceutical composition according to claim 1, wherein said zaleplon is present in a total amount chosen from 10, 15, 20, and 25 mg.

5. A pharmaceutical composition according to claim 1, wherein the amount of zaleplon present in each component ranges from about 30% to about 70%.

6. A pharmaceutical composition comprising:
a) a first component comprising zaleplon or a pharmaceutically acceptable salt thereof;
b) a second component comprising at least one particle, wherein said at least one particle comprises a core and at least one coating over said core, said core comprising zaleplon or a pharmaceutically acceptable salt thereof, and said at least one coating comprising at least one pharmaceutically acceptable polymer that is soluble at a pH greater than or equal to about 5.5; and
c) a third component comprising at least one particle, wherein said at least one particle comprises a core and at least one coating over said core, said core comprising zaleplon or a pharmaceutically acceptable salt thereof, and said at least one coating comprising at least one pharmaceutically acceptable polymer that is soluble at a pH greater than or equal to about 6.0.

7. A pharmaceutical composition according to claim 6, wherein the amount of zaleplon present in each component ranges from about 15% to about 50%.

8. A pharmaceutical composition comprising:
a) a first component comprising zaleplon or a pharmaceutically acceptable salt thereof;
b) a second component comprising at least one particle, wherein said at least one particle comprises a core and at least one coating over said core, said core comprising zaleplon or a pharmaceutically acceptable salt thereof, and said at least one coating comprising at least one pharmaceutically acceptable polymer that is soluble at a pH greater than or equal to about 5.5; and
c) a third component comprising at least one particle, wherein said at least one particle comprises a core and at least one coating over said core, said core comprising zaleplon or a pharmaceutically acceptable salt thereof, and said at least one coating comprising at least one pharmaceutically acceptable polymer that is soluble at a pH greater than or equal to about 7.0.

9. A pharmaceutical composition according to claim 8, wherein the amount of zaleplon present in each component ranges from about 15% to about 50%.

10. A pharmaceutical composition comprising:
a) a first component comprising zaleplon or a pharmaceutically acceptable salt thereof;
b) a second component comprising at least one particle, wherein said at least one particle comprises a core and at least one coating over said core, said core comprising zaleplon or a pharmaceutically acceptable salt thereof, and said at least one coating comprising at least one pharmaceutically acceptable polymer that is soluble at a pH greater than or equal to about 6.0; and
c) a third component comprising at least one particle, wherein said at least one particle comprises a core and at least one coating over said core, said core comprising zaleplon or a pharmaceutically acceptable salt thereof, and said at least one coating comprising at least one pharmaceutically acceptable polymer that is soluble at a pH greater than or equal to about 7.0.

11. A pharmaceutical composition according to claim 10, wherein the amount of zaleplon present in each component ranges from about 15% to about 50%.

12. A pharmaceutical composition comprising:
a) a first component comprising a short-acting hypnotic or a pharmaceutically acceptable salt thereof; and
b) a second component comprising at least one particle, wherein said at least one particle comprises a core and at least one coating over said core, said core comprising a short-acting hypnotic or a pharmaceutically acceptable salt thereof, and said at least one coating comprising at least one pharmaceutically acceptable polymer that is soluble at a pH greater than or equal to about 5.5.

13. A pharmaceutical composition according to claim 12, wherein said short-acting hypnotic is chosen from zaleplon, zopiclone, triazolam, temazepam, brotizolam, alimemazine, or zolpidem, and combinations thereof.

14. A pharmaceutical composition according to claim 12, wherein said at least one polymer is soluble at a pH greater than or equal to about 6.0.

15. A pharmaceutical composition according to claim 12, wherein said at least one polymer is soluble at a pH greater than or equal to about 7.0.

16. A pharmaceutical composition comprising:
a) a first component comprising a short-acting hypnotic or a pharmaceutically acceptable salt thereof;
b) a second component comprising at least one particle, wherein said at least one particle comprises a core and at least one coating over said core, said core comprising a short-acting hypnotic or a pharmaceutically acceptable salt thereof, and said at least one coating comprising at least one pharmaceutically acceptable polymer that is soluble at a pH greater than or equal to about 5.5; and
c) a third component comprising at least one particle, wherein said at least one particle comprises a core and at least one coating over said core, said core comprising a short-acting hypnotic or a pharmaceutically acceptable salt thereof, and said at least one coating comprising at least one pharmaceutically acceptable polymer that is soluble at a pH greater than or equal to about 6.0.

17. A pharmaceutical composition according to claim 16, wherein said short-acting hypnotic is chosen from zaleplon, zopiclone, triazolam, temazepam, brotizolam, alimemazine, or zolpidem, and combinations thereof.

18. A pharmaceutical composition comprising:
a) a first component comprising a short-acting hypnotic or a pharmaceutically acceptable salt thereof;
b) a second component comprising at least one particle, wherein said at least one particle comprises a core and at least one coating over said core, said core comprising a short-acting hypnotic or a pharmaceutically acceptable salt thereof, and said at least one coating comprising at least one pharmaceutically acceptable polymer that is soluble at a pH greater than or equal to about 5.5; and
c) a third component comprising at least one particle, wherein said at least one particle comprises a core and at least one coating over said core, said core comprising a short-acting hypnotic or a pharmaceutically acceptable salt thereof, and said at least one coating comprising at least one pharmaceutically acceptable polymer that is soluble at a pH greater than or equal to about 7.0.

19. A pharmaceutical composition according to claim 18, wherein said short-acting hypnotic is chosen from zaleplon, zopiclone, triazolam, temazepam, brotizolam, alimemazine, or zolpidem, and combinations thereof.

20. A pharmaceutical composition comprising:
a) a first component comprising a short-acting hypnotic or a pharmaceutically acceptable salt thereof;
b) a second component comprising at least one particle, wherein said at least one particle comprises a core and at least one coating over said core, said core comprising a short-acting hypnotic or a pharmaceutically acceptable salt thereof, and said at least one coating comprising at least one pharmaceutically acceptable polymer that is soluble at a pH greater than or equal to about 6.0; and
c) a third component comprising at least one particle, wherein said at least one particle comprises a core and at least one coating over said core, said core comprising a short-acting hypnotic or a pharmaceutically acceptable salt thereof, and said at least one coating comprising at least one pharmaceutically acceptable polymer that is soluble at a pH greater than or equal to about 7.0.

21. A pharmaceutical composition according to claim 20, wherein said short-acting hypnotic is chosen from zaleplon, zopiclone, triazolam, temazepam, brotizolam, alimemazine, or zolpidem, and combinations thereof.

22. A pharmaceutical composition according claim 12, wherein said short-acting hypnotic is present in a total amount chosen from 1, 5, 10, 15, 20, and 25 mg.

23. A method for treating sleep disorders comprising administering to a patient in need of such treatment an effective amount of a pharmaceutical composition comprising:
a) a first component comprising zaleplon or a pharmaceutically acceptable salt thereof; and
b) a second component comprising at least one particle, wherein said at least one particle comprises a core and at least one coating over said core, said core comprising zaleplon or a pharmaceutically acceptable salt thereof, and said at least one coating comprising at least one pharmaceutically acceptable polymer that is soluble at a pH greater than or equal to about 5.5.

24. The method according to claim 23, wherein the at least one pharmaceutically acceptable polymer is soluble at a pH greater than or equal to about 6.0.

25. The method according to claim 23, wherein the at least one pharmaceutically acceptable polymer is soluble at a pH greater than or equal to about 7.0.

26. A method of treating sleep disorders according to claim 23, wherein said sleep disorders are acute.

27. A method of treating sleep disorders according to claim 23, wherein said sleep disorders are chronic.

28. A method for treating sleep disorders comprising administering to a patient in need of such treatment an effective amount of a pharmaceutical composition comprising:
a) a first component comprising zaleplon or a pharmaceutically acceptable salt thereof;
b) a second component comprising at least one particle, wherein said at least one particle comprises a core and at least one coating over said core, said core comprising zaleplon or a pharmaceutically acceptable salt thereof, and said at least one coating comprising at least one pharmaceutically acceptable polymer that is soluble at a pH greater than or equal to about 5.5; and
c) a third component comprising at least one particle, wherein said at least one particle comprises a core and at least one coating over said core, said core comprising zaleplon or a pharmaceutically acceptable salt thereof, and said at least one coating comprising at least one pharmaceutically acceptable polymer that is soluble at a pH greater than or equal to about 6.0.

29. A method for treating sleep disorders according to claim 28, wherein said at least one pharmaceutically acceptable polymer on said second component is soluble at a pH greater than or equal to about 6.0, and said at least one pharmaceutically acceptable polymer on said third component is soluble at a pH greater than or equal to about 7.0.

30. A method of treating sleep disorders according to claim 28, wherein said sleep disorders are acute.

31. A method of treating sleep disorders according to claim 28, wherein said sleep disorders are chronic.

32. A method of inducing sleep in a patient, comprising administering to said patient an effective sleep-inducing amount of a composition according to claim 1.

33. A method of prolonging sleep in a patient, comprising administering to said patient an effective sleep-prolonging amount of a composition according to claim 1.

34. A method of treating sleep disorders according to claim 23, wherein said composition is in a form whereby it can be sprinkled on said patient's food and ingested by said patient.

35. A method of inducing sleep in a patient, comprising administering to said patient an effective sleep-inducing amount of a composition according to claim 12.

36. A method of prolonging sleep in a patient, comprising administering to said patient an effective sleep-prolonging amount of a composition according to claim 12.

37. The method of claim 23, wherein said patient does not experience a hangover effect.
